(19) United States
(12) Patent Application Publication
(10) Pub. No.: US 2011/0243988 A1
(43) Pub. Date: Oct. 6, 2011

(54) Methods and Compositions for Stabilization of a Virus Vaccine

(75) Inventors: Satoshi Ohtake, Milpitas, CA (US); Vu Truong-Le, Campbell, CA (US); Luisa Yee, Campbell, CA (US); Russell A. Martin, (US); David Lechuga-Ballesteros, San Jose, CA (US)

(73) Assignee: Aridis Pharmaceuticals

(21) Appl. No.: 12/896,165

(22) Filed: Oct. 1, 2010

Related U.S. Application Data
(60) Provisional application No. 61/247,860, filed on Oct. 1, 2009.

(51) Int. Cl. A61K 39/165; A61K 39/00; A61K 39/12; A61K 39/02; A61K 39/112; A61K 39/235; A61K 39/15; A61P 37/02

(52) U.S. Cl. 424/212.1; 424/184.1; 424/204.1; 424/234.1; 424/258.1; 424/233.1; 424/215.1

(57) ABSTRACT

This invention provides compositions and methods for stabilizing a live attenuated virus in dried formulations. In particular, compositions and methods of preparing a dried vaccine are provided that stabilize the viability of live vaccines such as measles and adenovirus at room temperature.

This application claims priority from U.S. Provisional Ser. No. 61/247,860 filed Oct. 1, 2009. This U.S. Provisional application is incorporated herein by reference. This application also claims priority from, and incorporates by reference, in its entirety, U.S. patent application Ser. No. 12/880,213 (filed Sep. 13, 2010), entitled Formulation for Room Temperature Stabilization of a Live Attenuated Bacterial Vaccine.

### FIELD OF THE INVENTION

The invention is a method to stabilize live virus vaccines, such as measles virus vaccine, using a combination of specific formulations and processing methods, including but not limited to spray drying, freeze drying, and foam drying.

### BACKGROUND OF THE INVENTION

World Health Organization (WHO) statistics indicate that more than three quarters of a million children die each year from measles. The cause of death is predominantly measles pneumonia, making it one of the most common respiratory diseases leading to death in children. Although the measles vaccines have been available since the early 1960s, they are regarded as one of the more unstable live vaccines that have been approved for human use. There is a desire for increased heat stability, especially in the developing world where transport, storage, and administration costs (mainly due to the need of continuous refrigeration, also referred to as the “cold chain”) represent a significant portion of the product cost. The current WHO requirements for heat stability employs two indices: 1) the vaccine should retain at least 3 Log₁₀ live virus particles in each human dose at the end of incubation at 37° C. for seven days; and 2) the virus titer should not have decreased by more than 1 Log₁₀ during storage.

Various attempts, involving both formulation development and process optimization, have been conducted to enhance the stability of a live measles vaccine. In U.S. Pat. No. 4,337,242, “Vaccine Stabilizer Containing L-Glutamic Acid and L-Arginine,” issued to Markus and McAleer, formulation components were described that stabilize a liquid live measles vaccine. The formulation included hydrolyzed gelatin (M.W. approx. 3,000 daltons) at 3-4.2% (w/v), sucrose at 14-26% (w/v), L-glutamic acid at 0.7-1.4% (w/v), L-arginine at 1.5-2.6% (w/v), in physiologically acceptable acidic buffer effective in maintaining the formulation pH between 6 and 6.5. The storage stability at 37° C. varied widely, ranging from about 0.3 to about 1.6 Log TCID₅₀ titer loss after 24 hours of storage. TCID is Tissue Culture Infective Doses. In U.S. Pat. No. 4,985,244, “Stabilized Live Attenuated Vaccine and Its Production”, issued to Makino, et al., formulation components to stabilize a live attenuated vaccine consisting of measles, mumps or rubella virus were described. The optimized formulation contained lactose at 2.5-5% (w/v), saccharose at 2.5-5% (w/v), D-sorbitol at 1.8-2% (w/v), sodium glutamate at about 0.1% (w/v), and gelatin hydrolyzate (M.W. approx. 35,000 daltons) at 2-3% (w/v). The formulated vaccine was subsequently lyophilized and stored at a high temperature. After 1 week of storage, the measles titer decreased by 0.2 Log₁₀ and 1.6 Log₁₀ at 37° C. and 45° C., respectively. As the study was terminated after 1 week of storage, the stability of the lyophilized vaccine upon extended storage is unknown, as well as their storage stability at lower temperatures. Furthermore, the effectiveness of the described formulation compositions to stabilize the measles virus employing other processing methods, e.g. spray drying, is unknown.

The storage stability of several other freeze dried measles vaccines at 37° C. is shown in Table 1 (Peetermans, J., et al (1978) Develop Biol Standard 41, 259-264). Most of the vaccines lose 1 Log₁₀ in virus titer within 1 week of storage at 37° C., failing to meet the WHO requirements. Furthermore, as freeze dried vaccines are administered after reconstitution and are typically prepared in a multi-dose format the stability of vaccine potency is highly dependent on the quality of cold-chain maintenance and the time between reconstitution and injection. The current route of administration also requires trained medical personnel and is associated with specific risk factors, such as the re-use or unsafe disposal of needles and syringes. These are compelling reasons to warrant an improved formulation and an alternate route for measles vaccine administration, e.g. pulmonary route.

TABLE 1
Stability of freeze dried measles vaccine

| Source/vaccine | T (° C.) | time | | Loss (log₁₀) |
|---|---|---|---|---|
| Edmonston Strain | 36 | 5 | days | 2.0 |
| Commercial Schwarz strain | 37 | 5 | days | 1.6 |
| L-16 strain | 35 | 4 to 5 days | | 1.0 |
| Mevilin-L | 35 | 1 | week | 0.7 |
| Attenuvax | 37 | 1 | week | 0.7 and 0.9 |
| Emonston-Zagreb | 37 | 7 | days | 1.0 |
| Rimevax 1^{st} generation | 37 | 7 | days | 0.78 |
| Rimevax 2^{nd} generation | 37 | 14 | days | 0.43 |

Compared to liquid formulations, solid formulations have multiple advantages such as avoidance of freeze-thaw stress, prevention of agitation/shear-induced aggregation, and increased ease in shipping and distribution. Furthermore, solid formulations decrease molecular motions and water-involved degradation reactions, which often results in improved stability and longer shelf-life of biopharmaceuticals. Drying techniques, e.g. spray drying, foam drying, and freeze drying, may also be employed to produce inhalable vaccine powders intended for pulmonary delivery. In case of foam and freeze drying, an additional processing step involving milling of the foam film or lyophilized cake, respectively, may be required to produce a flowable powder.

Some of the key considerations involved in preparing dry vaccine formulations include the exposure of virus particles to various thermal and mechanical stresses and the selection of excipients to minimize those damages. Furthermore, the formulation components must be compatible with the processing method chosen, e.g. avoidance of crystallization of buffer components or stabilizers, and collapse of glassy matrix for a freeze drying process. Depending on the freezing rate and the buffer component(s) chosen, the occurrence of salt crystallization and the rate of its formation are affected, potentially leading to pH change that may be detrimental to the stability of the labile biomolecule (Pikal-Cleland, K. A., et al (2000) Archives of Biochemistry and Biophysics 384, 398-406). In addition, with selective crystallization, the effectiveness of excipients as stabilizers is lost and the concentration of the remaining, unfrozen formulation components increases, which may have an additional impact on the stability of the biomolecule (Izutsu, K., et al (1993) Pharmaceutical Research 10, 1232-1237; Randolph, T. W. (2000) Journal of Pharmaceutical Sciences 86, 1198-1203). In case of spray drying, the extent of measles virus enrichment on the surface of the spray dried particles is expected to have a large influence on the storage stability of the virus (Abdul-Fattah, A. M., et al (2007) Pharmaceutical Research 24, 715-727). To mitigate the damage, surface active molecules may be added, in addition to modifying the spray drying condition.

Freeze drying and spray drying are two of the widest used methods of drying active pharmaceutical ingredient (API) solutions in the pharmaceutical industry. Freeze drying has been employed to produce several commercial API products, including measles vaccines (Table 1). The challenge of employing a freeze drying process on a labile biomolecule include the exposure of the virus to low temperature, adsorption of viral particles to ice crystal surface, and dehydration stress, to name a few. In addition, for pulmonary delivery applications, the lyophilized samples typically require milling to produce a flowable powder with the required aerodynamic properties, which may further stress the virus. Spray drying provides advantages of offering high volume product throughput (>5,000 lb/hr) and reduced manufacturing times over other protein preservation/drying technologies such as freeze drying. The challenge of using spray drying to stabilize thermally labile APIs, such as viruses, involves the control of three key areas: atomization conditions, drying conditions, and resultant solid state properties of the dried material. For example, during atomization, the process of breaking up the liquid stream into fine droplets can involve excessive shear stress, surface tension, and pressure applied to the product, leading to loss of bioactivity. Another challenge involves the control of droplet drying rate and its interplay with the components within each droplet. Depending on the process parameters, e.g. the drying rate and the droplet size, and the formulation components, e.g. surface activity and molecular size (i.e. diffusion rate), it is possible to manipulate the properties of the resultant dried particles, which include the particle size, surface composition, and surface morphology. This control is important, as the storage stability of the biopharmaceutical is generally influenced by the degree of its surface enrichment, as well as by the porosity and surface area of the spray dried particles.

One of the main reasons for creating a flowable dry powder is to facilitate the preparation of biopharmaceuticals for pulmonary delivery. However, spray dried powders may also be incorporated into a variety of other dosage formats, including but not limited to mucoadhesive thin films (Cui, Z. and Mumper, R. J. (2002) Pharmaceutical Research 19, 1901-1906), transdermal skin patches (Glenn, G. M. and Kenney, R. T. (2006) Curr. Top. Microbiol. Immunol. 304, 247-268), controlled release polymer matrices (Gupta, R. K., et al (1998) Adv. Drug Del. Rev. 32, 225-246), enteric coated tablets (Wilding, I. R., et al (1994) Pharmacol. Ther. 62, 97-124), and wafers (Rak, S., et al (2007) Qual. Life Res. 16, 191-201), thereby increasing the options available for drug delivery.

The key attributes of the present invention involve the identification of unique formulation combinations and the application of a well controlled dehydration technique, e.g. spray drying, freeze drying, and foam drying, that allow for the production of stable measles virus in a dry powder format. The availability of a dry measles virus vaccine may enhance the possibility to allow their storage in remote parts of the world, where low temperature transport and storage are not feasible. Furthermore, a more convenient method of mass vaccination by developing simple, convenient, easy-to-administer dosage presentations for a measles vaccine candidate is needed. The present invention provides these and other features that will be apparent upon review of the following.

### SUMMARY OF THE INVENTION

The invention discloses a novel formulation that is suitable for stabilization of dry powder live virus vaccines, such as measles virus, produced through spray drying, freeze drying, and/or foam drying. The stabilizing formulation components include, but are not limited to, a polyol, a polymer, a surfactant, a plasticizer, a divalent cation, an amino acid, and a buffer. Polyols and polymers, such as sucrose, trehalose, and human serum albumin, are included in the formulation to act as a stabilizer for the virus. Stabilization is widely accepted to occur through the replacement of lost hydrogen bonds due to dehydration and/or through the formation of a glassy matrix (Crowe, J. H., et al (1984) Science 223, 701-703; Koster, K. L., et al (2000) Biophysical Journal 78, 1932-1946). Polyol concentration in the virus vaccine-containing formulation may range from about 5% to about 70% (w/v), while the polymer concentration may range from about 0.1% to about 20% (w/v). Surfactants, such as Pluronic F68, are included to decrease the surface tension of the atomized droplets and to displace the virus molecules from the surface of the atomized droplets. In addition, surfactants may be incorporated into the present invention to increase the solubility of other formulation components. Surfactant concentration may comprise at most 2% by weight of said virus vaccine-containing formulation. Buffering components, such as phosphate and citrate, are included to control the pH of the virus vaccine-containing solution, as well as to adjust the solution osmolarity. The buffer concentration may range from about 5 mM to about 2M, with the pH of the solution adjusted to a range from about pH4 to about pH10. A plasticizer, such as glycerol, is included to increase the interaction of the glassy matrix with the virus vaccine upon dehydration, thereby enhancing storage stability (Cicerone, M. T., et al, U.S. Pat. No. 7,101,693). The concentration of plasticizer in the present invention may comprise at most 5% by weight of the formulation. Divalent cations and certain amino acids, such as ZnCl₂ and arginine, are included to stabilize the viral and to adjust the pH and the osmolarity of the solution. The divalent cation concentration may range from about 0.1 mM to about 100 mM, and the amino acid concentration may range from about 0.1% to about 10% (w/v). In a preferred embodiment of the current invention, dry powder virus vaccine is prepared by spray drying.

In preferred embodiments, the vaccine formulation includes a live vaccine at a titer ranging from about 3 to about 9 Log TCID₅₀/mL, the polyol is a mixture of sucrose and trehalose present in concentration ranging from about 5% to about 20% (w/v) for each component, the buffer is potassium phosphate ranging in concentration from about 10 mM to about 100 mM adjusted to pH of about 6 to 7, the divalent cation is a mixture of calcium chloride and zinc chloride present at a concentration ranging from about 1 mM to about 10 mM for each component, the amino acid is arginine present at a concentration ranging from about 1% to about 8% (w/v), the plasticizer is glycerol ranging in concentration from about 0.1% to about 5% by weight of said formulation, and the surfactant is a block copolymer of polyethylene and polypropylene glycol present at a concentration ranging from about 0.04% to 0.2% by weight of said formulation. In a more preferred embodiment, the said formulation further comprises of human serum albumin at a concentration ranging from about 1% to about 10% (w/v).

In another aspect of the present invention, the virus vaccine-containing compositions may be prepared as a dry powder. Dry powder production can be conducted employing a variety of methods known to those skilled in the art, which includes, but is not limited to foam drying, freeze drying, spray drying, spray freeze drying, fluidized bed drying, supercritical fluid assisted drying, and vacuum drying. Spray drying is most preferable for use in the present invention.

The spray drying process of the present invention employs an ultrasonic atomization nozzle to produce dry powder particles that can be room temperature stable and exhibit appropriate powder properties for deep lung delivery as well as for fabrication into other dosage formats such as oral wafers, oral thin films, capsules, tablets, etc. In this method, a solution of virus vaccine is first formulated with stabilizing excipients, as described above, and then atomized from a nozzle using pressurized gas, with or without an organic solvent serving as a liquid modifier. The atomizing gas can be air or any other gases, preferably air, nitrogen, CO₂ at or near supercritical state. The atomized virus vaccine is caused to dry into powder particles by infusing a stream of dry, heated gas co-current to the spray plume. The gas used to evaporate the atomized solution i.e. drying gas, is typically heated and can be air, nitrogen, argon, or the like. The spray drying equipment can be any commercially available spray dryers and nozzles, but preferably with commercially available ultrasonic nozzles, more preferably ultrasonic nozzles that are non-piezoelectric and operate at low pressure range.

The invention provides a dry vaccine composition comprising: (a) A virus vaccine at a titer ranging from about 3 Log TCID₅₀/mL to about 9 Log TCID₅₀/mL; (b) a polyol at a concentration ranging from about 5% to about 70% (w/v); (c) a pharmaceutically acceptable buffer ranging from about 5 mM to about 2M; (d) a divalent cation ranging in concentration from about 0.1 mM to about 100 mM; (d) at least one component selected from the group consisting of an amino acid, a plasticizer, a polymer, or a surfactant. In another aspect, the invention comprises the above composition, wherein the virus vaccine is one of rotavirus, adenovirus, mumps virus, rubella virus, polio virus, influenza virus, parainfluenza virus, vaccinia virus, respiratory syncytial virus, herpes simplex virus, SARS virus, corona virus family members, cytomegalovirus, human metapneumovirus, filovirus, and Epstein-Bar virus. In yet another aspect, the invention comprises one of the above compositions, wherein the virus vaccine is live measles virus vaccine. Regarding polyols, the invention comprises one of the above compositions, wherein a polyol is selected from the group consisting of sucrose, trehalose, sorbose, melezitose, sorbitol, stachyose, raffinose, fructose, mannose, maltose, lactose, arabinose, xylose, ribose, rhamnose, galactose, glucose, mannitol, myo-inositol, xylitol, erythritol, threitol, sorbitol, glycerol, L-glyconate, dextrose, fucose, polyaspartic acid, inositol hexaphosphate (phytic acid), sialic acid, N-acetylneuraminic acid-lactose, and their mixtures thereof. Moreover, what is contemplated is an embodiment, wherein the polyol is sucrose present at a concentration ranging from about 5% to about 40% (w/v); and in other aspect, wherein the polyol is trehalose present at a concentration ranging from about 5% to about 40% (w/v); and in yet another aspect, wherein the polyol is a mixture of sucrose and trehalose.

Buffer embodiments of the present invention, without limitation, include one of the above compositions, wherein a pharmaceutically acceptable buffer is selected from the group consisting of potassium phosphate, sodium phosphate, sodium acetate, histidine, imidazole, sodium citrate, sodium succinate, ammonium bicarbonate, and a carbonate. Moreover, what is provided is the above composition, wherein the pharmaceutically acceptable buffer is potassium phosphate present at a concentration ranging from about 5 mM to about 200 mM.

Divalent cation embodiments are also contemplated, including one of the above compositions, wherein a divalent cation is selected from the group consisting of a pharmaceutically acceptable salt of magnesium, zinc, calcium, manganese, and their combinations thereof, and also wherein the divalent cation is calcium present at a concentration ranging from about 1 mM to about 5 mM, and also wherein the divalent cation is zinc present at a concentration ranging from about 1 mM to about 5 mM, and in yet another aspect, wherein the divalent cation is a mixture of calcium and zinc.

Amino acid embodiments of one or all of the above examples are provided, wherein an amino acid can be alanine, arginine, methionine, serine, lysine, histidine, glutamic acid, and their combinations thereof, and also optionally, wherein an amino acid is present at a concentration ranging from about 0.1% to about 10% (w/v), and also optionally, wherein the amino acid is arginine present at a concentration ranging from about 1% to about 8% (w/v).

Plasticizer embodiments of one or more of the above compositions are encompassed, for example, wherein a plasticizer is selected from the group consisting of glycerol, dimethylsulfoxide (DMSO), propylene glycol, ethylene glycol, oligomeric polyethylene glycol, sorbitol, and their combinations thereof, and also, wherein a plasticizer is present at a concentration ranging from about 0.1% to about 5% by weight of said formulation, and also wherein the plasticizer is glycerol at a concentration not exceeding 5% by weight of said formulation.

Polymer embodiments are provided, for example, wherein a polymer is selected from the group consisting of gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, human serum albumin, lactalbumin hydrolysate, and their combinations thereof. In additional aspects of the polymer embodiments of the present invention, what is provided is a polymer is present at a concentration ranging from about 0.1% to about 20% (w/v), and also, wherein the polymer is human serum albumin present at a concentration ranging from about 1% to about 10% (w/v).

Surfactant embodiments of any one of the above compositions are provided, for example, compositions wherein a surfactant selected from the group consisting of polyethylene glycol, polypropylene glycol, polyethylene glycol/polypropylene glycol block copolymers, polyethylene glycol alkyl ethers, polyethylene glycol sorbitan monolaurate, polypropylene glycol alkyl ethers, polyethylene glycol/polypropylene glycol ether block copolymers, polyoxyethylenesorbitan monooleate, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde and phenol, lignin-sulfite waste liquor, alkyl phosphates, quaternary ammonium compounds, amine, oxides, and betaines, and in yet another aspect, wherein a surfactant is present at a concentration ranging from about 0.01% to about 2% by weight of said formulation, and in still another aspect, wherein the surfactant is block copolymers of polyethylene and polypropylene glycol at a concentration ranging from about 0.02% to about 0.5% by weight of said formulation.

Acidity and alkalinity embodiments of any one of the above compositions, additionally include, wherein the buffer comprises a pH ranging from about pH 4 to about pH 10, and also wherein the buffer comprises a pH ranging from about pH 6 to about pH 8, and also, wherein the buffer comprises a pH of about pH 6 to about pH 7.

Process embodiments of any one of the above compositions include, but are not limited to, a dry vaccine composition prepared by spray drying, a dry vaccine composition prepared by freeze drying, and also, a dry vaccine composition prepared by a process comprising:

preparing a formulation containing a live attenuated strain of a measles virus; reducing pressure on the formulation, whereby a foam is formed, the foam frozen, and ice is sublimated, thereby providing a lyophilized dry foam composition. Moreover, what is encompassed is a dry vaccine composition comprising: (a) live attenuated strain of a measles virus at a titer ranging from about 3 Log TCID₅₀/mL to about 9 Log TCID₅₀/mL; (b) a mixture of sucrose and trehalose, each present at a concentration ranging from about 5% to about 20% (w/v); (c) potassium phosphate at a concentration ranging from about 10 mM to about 100 mM adjusted to pH of about 6 to 7; (d) a mixture of calcium chloride and zinc chloride, each present at a concentration ranging from about 1 mM to about 10 mM; (e) arginine at a concentration ranging from about 1% to about 8% (w/v); (f) glycerol at a concentration ranging from about 0.1% to about 5% by weight of said formulation; (g) human serum albumin at a concentration ranging from about 1% to about 10% (w/v). What is also encompassed, is the above dry vaccine composition that is prepared by spray drying, or that is prepared by freeze-drying, or that is prepared by a process comprising: preparing a formulation containing a virus vaccine;

reducing pressure on the formulation, whereby a foam is formed, the foam frozen, and ice is sublimated, thereby providing a lyophilized dry foam composition.

The present invention provides various composition embodiments, for example, wherein the virus vaccine is selected from a list that includes rotavirus, adenovirus, mumps virus, rubella virus, polio virus, influenza virus, parainfluenza virus, vaccinia virus, respiratory syncytial virus, herpes simplex virus, SARS virus, corona virus family members, cytomegalovirus, human metapneumovirus, filovirus, and Epstein-Bar virus. Other encompassed composition embodiments encompass a virus vaccine that is a live measles virus vaccine. Still other composition embodiments have a polyol is selected from the group consisting of sucrose, trehalose, sorbose, melezitose, sorbitol, stachyose, raffinose, fructose, mannose, maltose, lactose, arabinose, xylose, ribose, rhamnose, galactose, glucose, mannitol, myo-inositol, xylitol, erythritol, threitol, sorbitol, glycerol, L-glyconate, dextrose, fucose, polyaspartic acid, inositol hexaphosphate (phytic acid), sialic acid, N-acetylneuraminic acid-lactose, and their mixtures thereof. And still other composition embodiments have a polyol that is sucrose present at a concentration ranging from about 5% to about 40% (w/v). In more composition embodiments, the polyol is trehalose present at a concentration ranging from about 5% to about 40% (w/v). In still other composition embodiments, the polyol is a mixture of sucrose and trehalose. In yet other embodiments, what is encompassed is a pharmaceutically acceptable buffer is selected from the group consisting of potassium phosphate, sodium phosphate, sodium acetate, histidine, imidazole, sodium citrate, sodium succinate, ammonium bicarbonate, and a carbonate. Moreover, what is included is a pharmaceutically acceptable buffer is potassium phosphate present at a concentration ranging from about 5 mM to about 200 mM. Also contemplated is the above composition, wherein a divalent cation is selected from the group consisting of a pharmaceutically acceptable salt of magnesium, zinc, calcium, manganese, and their combinations thereof, and also the above composition, wherein the divalent cation is calcium present at a concentration ranging from about 1 mM to about 5 mM, and other composition embodiments wherein the divalent cation is zinc present at a concentration ranging from about 1 mM to about 5 mM, and yet other composition embodiments, wherein the divalent cation is a mixture of calcium and zinc, and yet other composition embodiments, wherein an amino acid is selected from the group consisting of alanine, arginine, methionine, serine, lysine, histidine, glutamic acid, and their combinations thereof, and still more composition embodiments, wherein an amino acid is present at a concentration ranging from about 0.1% to about 10% (w/v), and, without limitation, more composition embodiments, for example, wherein the amino acid is arginine present at a concentration ranging from about 1% to about 8% (w/v), and also plasticizer embodiments, wherein a plasticizer is selected from the group consisting of glycerol, dimethylsulfoxide (DMSO), propylene glycol, ethylene glycol, oligomeric polyethylene glycol, sorbitol, and their combinations thereof, and still other plasticizer embodiments, wherein a plasticizer is present at a concentration ranging from about 0.1% to about 5% by weight of said formulation, and more plasticizer embodiments, wherein the plasticizer is glycerol at a concentration not exceeding 5% by weight of said formulation. Polymer embodiments are included in each of the above embodiments, for example, wherein a polymer is selected from the group consisting of gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, human serum albumin, lactalbumin hydrolysate, and their combinations thereof. In other polymer embodiments, a polymer is present at a concentration ranging from about 0.1% to about 20% (w/v). In yet further polymer embodiments, the polymer is human serum albumin present at a concentration ranging from about 1% to about 10% (w/v). Surfactant embodiments are included, and these can be selected from the group consisting of polyethylene glycol, polypropylene glycol, polyethylene glycol/polypropylene glycol block copolymers, polyethylene glycol alkyl ethers, polyethylene glycol sorbitan monolaurate, polypropylene glycol alkyl ethers, polyethylene glycol/polypropylene glycol ether block copolymers, polyoxyethylenesorbitan monooleate, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde and phenol, lignin-sulfite waste liquor, alkyl phosphates, quaternary ammonium compounds, amine, oxides, and betaines. In another aspect of a surfactant embodiemnt, a surfactant is present at a concentration ranging from about 0.01% to about 2% by weight of said formulation. What is further contemplated, with regard to surfactants, the surfactant is block copolymers of polyethylene and polypropylene glycol at a concentration ranging from about 0.02% to about 0.5% by weight of said formulation. Now, with regard to pH, what is encompassed is the above composition, wherein the buffer comprises a pH ranging from about pH 4 to about pH 10. Also, what is provided is the above compositions, wherein the buffer comprises a pH ranging from about pH 6 to about pH 8. And also, embodiments wherein the buffer comprises a pH of about pH 6 to about pH 7.

Process embodiments are included in the present invention. Without limitation, these include spray drying, freeze drying, and also a dry vaccine composition as set forth above, prepared by a process comprising: preparing a formulation containing a live attenuated strain of a measles virus; reducing pressure on the formulation, whereby a foam is formed, the foam frozen, and ice is sublimated, thereby providing a lyophilized dry foam composition. And also as set forth above, what is included in the present invention is a dry vaccine composition comprising:

(a) live attenuated strain of a measles virus at a titer ranging from about 3 Log TCID₅₀/mL to about 9 Log TCID₅₀/mL; (b) a mixture of sucrose and trehalose, each present at a concentration ranging from about 5% to about 20% (w/v); (c) potassium phosphate at a concentration ranging from about 10 mM to about 100 mM adjusted to pH of about 6 to 7; (d) a mixture of calcium chloride and zinc chloride, each present at a concentration ranging from about 1 mM to about 10 mM; (e) arginine at a concentration ranging from about 1% to about 8% (w/v); (f) glycerol at a concentration ranging from about 0.1% to about 5% by weight of said formulation; and (g) human serum albumin at a concentration ranging from about 1% to about 10% (w/v). Moreover, what is provided are dry vaccine compositions prepared by either spray drying, freeze drying, or by a process comprising: a) preparing a formulation containing a virus vaccine; reducing pressure on the formulation, whereby a foam is formed, the foam frozen, and ice is sublimated, thereby providing a lyophilized dry foam composition.

In divalent cation embodiments, in particular embodiments wherein the formulation is combined or mixed with a virus, the calcium in the formulation is under 20 mM, under 10 mM, under 5 mM, under 4 mM, under 3 mM, under 2 mM, under 1 mM, and the like. Or wherein zinc in the formulation is under 20 mM, under 10 mM, under 5 mM, under 4 mM, under 3 mM, under 2 mM, under 1 mM, and the like. What is provided, without limitation, a formulation wherein magnesium in the formulation is under 20 mM, under 10 mM, under 5 mM, under 4 mM, under 3 mM, under 2 mM, under 1 mM, and the like. And also, the formulation embodiments encompass embodiments wherein the total concentration of divalent cations in the formulation is under 40 mM, under 20 mM, under 10 mM, under 5 mM, under 4 mM, under 3 mM, under 2 mM, under 1 mM, and the like.

Preferred embodiments of the present invention are set forth. What is encompassed is a dry vaccine composition prepared with a formulation, the composition comprising: a) a biologically active sample; b) a polyol that, in the formulation, is about 5% to about 70% (w/v); c) a divalent cation that, in the formulation, is about 0.1 mM to about 100 mM; d) a plasticizer of 1% to 10% (w/w) in the formulation; and e) an amino acid, a polymer, or surfactant, or any combination thereof; wherein the dry vaccine composition is prepared by spray drying. Also provided is the above composition, wherein the biologically active sample is a virus at a titer ranging from about 3 log per mL to about 9 log per mL, wherein the titer is determined on the liquid-reconstituted vaccine or determined in the liquid form before processing to make the dry vaccine composition. In another aspect, what is provided is the above composition, wherein the biologically active sample is bacteria or an immunologically active oligopeptide or immunologically active polypeptide. In yet another aspect, what is provided is the above composition, wherein the biologically active sample is a bacterium, including an attenuated bacterium, and including Salmonella typhi, and also including sub-unit protein antigen, or that is aluminum-adjuvanted or that contains an oligopeptide or oligonucleotide that can induce an immune response. In an embodiment that guides the preparation of a virus embodiment, the invention contemplates,

the above composition, wherein the biological active sample is a virus with a measurable titer, wherein the titer is determined by tissue culture infective dose (TCID) assay or by fluorescent focus assay (FFA). In virus and buffer embodiments, what is encompassed is the above composition, wherein the virus is measles virus, and the formulation contains less than 50 mM pharmaceutically acceptable buffer, and where the virus is measles virus, and the formulation contains less than 50 mM potassium, and also where the virus is measles virus, and the formulation contains less than 10mM pharmaceutically acceptable buffer, and also where the virus is measles virus, and the formulation contains less than 10 mM potassium. In adenovirus embodiments, the optimal buffer for stability may differ from the optimal buffer for stability of a measles embodiment, and for compositions where the virus is adenovirus, what is provided is a formulation that contains greater than 100 mM potassium. Potassium for the adenovirus embodiment, or other viruses, can be greater than 50 mM, 75 mM, 100 mM, 125 mM, 150 mM, 175 mM, 200 mM, 225 mM, 250 mM, 275 mM, 300 mM, 350 mM, 400 mM, 450 mM, or greater than 500 mM, and the like. Other potassium embodiments, useful for adenovirus or other viruses, have formulations with potassium from 25-50 mM, 50-75 mM, 75-100 mM, 75-125 mM, 100-150 mM, 125-175 mM, 150-200 mM, 175-225 mM, 200-250 mM, 225-275 mM, 250-300 mM, 300-350 mM, 350-400 mM, 400-450 mM, 450500 mM, and the like.

Divalent cation embodiments encompass one or more of the above compositions, wherein the formulation contains between 0.1-10 mM calcium and 0.1-10 mM zinc. In embodiments having optimal or maximal stability, what is provided is the above composition, that is prepared with a first formulation that contains 0.1 mM to about 50 mM calcium and 0.1 mM to about 50 mM zinc, and wherein there is a second formulation that contains the same components, at the same concentrations, as the first formulation, but where the second formulation has no zinc, and where the stability of the dried vaccine composition prepared from the first formulation is at least 3.0-fold greater than that of a dried vaccine composition with the same virus, but prepared with the second formulation, and also wherein the fold greater is, at least 5.0-fold greater, and also, wherein the stability is process stability, and also wherein the stability is storage stability.

In another embodiment that provides optimal stability or maximal stability, what is provided is the above composition, that is prepared with a first formulation that contains 0.1 mM to about 50 mM calcium, and 0.1 mM to about 50 mM zinc, and wherein there is a second formulation that contains the same components, at the same concentrations, as the first formulation, but where the second formulation has no calcium, and where the stability of the dried vaccine composition prepared from the first formulation is at least 3.0-fold greater than that of a dried vaccine composition with the same virus, but prepared with the second formulation, or wherein the fold-greater is at least 5.0-fold greater, or wherein the stability is process stability, or wherein the stability is storage stability.

In a solids embodiment, what is provided is the above composition, where the formulation has greater than 10% solids and less than 40% solids, and also the above composition, where the formulation has greater than 15% solids and less than 40% solids, and also the above compositions, where the formulation has greater than 20% solids and less than 40% solids.

In more viral embodiments, what is provided is each of the above compositions, wherein the virus is a live attenuated virus, or wherein the virus is an enveloped virus, or wherein the virus is a non-enveloped virus, or wherein the virus is a measles virus or a rotavirus. In polyol aspects of the invention, what is provided is sucrose, trehalose, or a mixture of sucrose and trehalose. In divalent cation embodiments, what is provided is calcium, zinc, magnesium, or a combination thereof. In amino acid versions of the invention, what is embraced is alanine, arginine, methionine, serine, lysine, histidine, glutamic acid, or a combination thereof. Also, what is provided are examples, wherein the formulation comprises a pharmaceutically acceptable buffer. Plasticizer embodiments include glycerol, dimethylsulfoxide (DMSO), propylene glycol, ethylene glycol, oligomeric polyethylene glycol, sorbitol, or a combination thereof. Polymer embodiments include, gelatin, partially hydrolyzed gelatin, albumin, or a combination thereof. Surfactant embodiments of any one or more of the above compositions, include polyethylene glycol, polypropylene glycol or a surfactant with the chemical structure of Pluronic® F68.

Methods embodiments are also embraced by the present invention, such as, a method for preparing the composition of claim 1 that comprises combining or mixing a formulation with the virus, and then spray drying wherein the pressure (Pₐₜₘ) is less than 25 psi, or wherein the temperature (Tₒᵤₜ) is less than 60 degrees C., or wherein the pressure is less than 25 psi and the temperature is less than 60 degrees C., and also a method for preparing the composition of claim 1 that comprises combining or mixing a formulation with a virus, and then spray drying wherein the pressure (Pₐₜₘ) is about 10-20 psi, or wherein the temperature (Tₒᵤₜ) is about 30-50 degrees C., or wherein the pressure is about 10-20 psi and the temperature is about 30-50 degrees C.

In one particular example, the invention provides, a dry vaccine composition of claim 1, comprising: a) live attenuated strain of a measles virus at a titer ranging from about 3 Log TCID₅₀/mL to about 9 Log TCID₅₀/mL; b) a mixture of sucrose and trehalose, each present at a concentration ranging from about 5% to about 20% (w/v); c) potassium phosphate at a concentration ranging from about 10 mM to about 100 mM adjusted to pH of about 6 to 7;

d) a mixture of calcium chloride and zinc chloride, each present at a concentration ranging from about 1 mM to about 10 mM; e) arginine at a concentration ranging from about 1% to about 8% (w/v); f) glycerol at a concentration ranging from about 0.1% to about 5% by weight of said formulation; and g) human serum albumin at a concentration ranging from about 1% to about 10% (w/v).

Formulation embodiments of the present invention include a formulation of Table 3, Table 5, Table 6, Table 8, Table 9, Table 11, Table 13, Table 14, or Table 15.

A dry vaccine composition that embraces divalent cations, is a dry vaccine composition made from a virus and a first liquid formulation, wherein the first liquid formulation contains calcium and zinc, wherein the dry vaccine composition is a first dry vaccine composition, wherein the stability of the first dry vaccine is greater than the stability of a second dry vaccine, wherein the second dry vaccine is prepared with the same virus, same formulation components, and same formulation concentrations, as used for the first dry vaccine composition, except that the second liquid formulation does not contain any calcium, or does not contain any zinc. Also, what is embraced is the above dry vaccine composition, wherein the stability that is greater, is at least 3.0-fold greater than that of the second dry vaccine, and also, wherein the stability that is greater, is at least 5.0-fold greater than that of the second dry vaccine, and also wherein the virus is measles virus, and also wherein the first liquid formulation contains a pharmaceutically acceptable buffer, and also wherein the first liquid formulation contains one or more of a polyol, an amino acid, a plasticizer, a polymer, a surfactant, or any combination thereof, and also wherein the stability is process stability, and also wherein the stability is storage stability.

### Definitions

Unless otherwise defined herein or below in the remainder of the specification, all technical and scientific terms used herein have meanings commonly understood by those of ordinary skill in the art to which the present invention belongs. Although any methods and materials similar or equivalent to those described herein can be used in the practice for testing of the present invention, the preferred materials and methods are described herein. In describing and claiming the present invention, the following terminology will be used in accordance with the definitions set out below.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “a buffer” can include mixtures of buffers, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “about”, as used herein, indicates the value of a given quantity can include quantities ranging within 10% of the stated value, or optionally within 5% of the value, or in some embodiments within 1% of the value.

“Ambient” temperatures or conditions are those at any given time in a given environment. Typically, ambient room temperature is 22° C., ambient atmospheric pressure, and ambient humidity are readily measured and will vary depending on the time of year, weather conditions, altitude, etc.

“Dry” in the context of dried foam compositions, as well as those prepared by freeze drying and spray drying, refers to residual moisture content less than about 10%. Dried compositions are commonly dried to residual moistures of 5% or less, or between about 3% and 0.1%.

“Excipients” or “protectants” (including cryoprotectants and lyoprotectants) generally refer to compounds or materials that are added to ensure or increase the stability of the therapeutic agent during the dehydration processes, e.g. foam drying, spray drying, freeze drying, etc., and afterwards, for long term stability.

“Glass” or glassy state” or “glassy matrix” refers to a liquid that has lost its ability to flow, i.e. it is a liquid with a very high viscosity, wherein the viscosity ranges from 10¹⁰ to 10¹⁴ pascal seconds. It can be viewed as a metastable amorphous system in which the molecules have vibrational motion but have very slow rotational and translational components. As a metastable system, it is stable for long periods of time when stored well below the glass transition temperature. Because glasses are not in a state of thermodynamic equilibrium, glasses stored at temperatures at or near the glass transition temperature relax to equilibrium and lose their high viscosity. The resultant rubbery or syrupy, flowing liquid is often chemically and structurally destabilized. While a glass can be obtained by many different routes, it appears to be physically and structurally the same material by whatever route it was taken. The process used to obtain a glassy matrix for the purposes of the invention is generally a solvent sublimation and/or evaporation technique.

The “glass transition temperature” is represented by the symbol T_{g} and is the temperature at which a composition changes from a glassy or vitreous state to a syrup or rubbery state. Generally T_{g} is determined using differential scanning calorimetry (DSC) and is standardly taken as the temperature at which onset of the change of heat capacity (Cₚ) of the composition occurs upon scanning through the transition. The definition of T_{g} is always arbitrary and there is no present international convention. The T_{g} can be defined as the onset, midpoint or endpoint of the transition.

A “stable” formulation or composition is one in which the biologically active material therein essentially retains its physical stability and/or chemical stability and/or biological activity upon storage. Stability can be measured at a selected temperature for a selected time period. Trend analysis can be used to estimate an expected shelf life before a material has actually been in storage for that time period.

“Pharmaceutically acceptable” refers to those active agents, salts, and excipients which are, within the scope of sound medical judgment, suitable for use in contact with the tissues or humans and lower animals without undue toxicity, irritation, allergic response and the like, commensurate with a reasonable benefit/risk ratio, and effective for their intended use.

### BRIEF DESCRIPTION OF THE DRAWINGS

FIG. 1 shows the effect of starting measles virus titer on the stability of measles virus stored at 37° C. Spray dried measles virus with a starting titer of 5.4 Log TCID₅₀/mL is indicated in the figure by white bars, while that with a starting titer of 4.3 Log TCID₅₀/mL is indicated in the figure by gray bars.

FIG. 2 shows the stability of spray dried measles formulations stored at 37° C. The formulation components are indicated in Table 5; the symbols correspond to formulation M6 (▪), M7 (), M8 (▴), M9 (▾), and M10 (♦).

FIG. 3 shows the effect of proteins on the stability of spray dried measles stored at 37° C. The formulation components are shown in Table 8; the symbols correspond to formulation M13 (▪), M14 (), and M15 (▴).

FIG. 4 shows the effect of surfactant addition on the storage stability of spray dried measles. The stability of measles virus, formulated with and without a surfactant (represented by closed and open symbols, respectively), is shown following storage at 4° C. (), 25° C. (▴), and 37° C. (▪) for the indicated amount of time.

FIG. 5 compares the effects of various drying processes on the recovery and 1 week storage stability of dry measles virus at 37° C. Titer decrease for dried measles are indicated as: spray drying (white bars), spray drying followed by secondary drying (black bars), freeze drying (gray bars), and foam drying (striped bars).

FIG. 6 shows stabilization of antacid containing monovalent bovine rotavirus (BRV). The formulation used in the top bar contained sucrose+Zn+Ca+protein, next bar (sucrose+Zn+Ca), next bar (sucrose+Ca), and bottom bar (sucrose).

### DETAILED DESCRIPTION

The present invention is the result of extensive experimentation to identify new combinations of vaccine formulation constituents and methods of preparing stable dry virus vaccine, such as measles virus.

In one embodiment of dry virus vaccine formulations, the viability of live attenuated virus is enhanced in the presence of specific combinations of pharmaceutically acceptable excipients employing an optimized drying process. For example, the viability of dry powder virus vaccine produced by spray drying can be extended during storage at room temperature in a formulation containing any of: 1) a polyol; 2) a divalent cation; 3) an amino acid; 4) a soluble polymer; 5) a plasticizer; 6) a surfactant; and 7) a pharmaceutically acceptable buffer.

Polyol embodiments are contemplated. Live virus vaccine, such as measles vaccine, was found to be more stable in the presence of substantial amounts of a polyol, such as a substantially water soluble sugar. Furthermore, the polyol may be included to aid in certain drying processes, e.g. spray drying, by increasing the solution viscosity, and in freeze drying, by acting as a bulking agent. In addition, polyols can be included to modify the osmolarity of the measles virus-containing solution. In one aspect, the sugar is present in an amount ranging from about 5% to 70% (w/v). In preferred embodiments, the sugar, whether as a single component or as a mixture of two or more components, is present in the formulation in the range between 5% and 70%, 10% and 50%, 15% and 30%, or about 20% (w/v). In preferred embodiments, the sugar is present in the formulation at a concentration ranging from about 15% to about 30% (w/v).

More preferred polyols include, e.g., sucrose, trehalose, sorbose, melezitose, sorbitol, stachyose, raffinose, fructose, mannose, maltose, lactose, arabinose, xylose, ribose, rhamnose, galactose, glycose, mannitol, myo-inositol, xylitol, erythritol, threitol, sorbitol, glycerol, L-glyconate, dextrose, fucose, polyaspartic acid, inositol hexaphosphate (phytic acid), sialic acid, N-acetylneuraminic acid-lactose, and their combinations thereof. In a typical embodiment, the formulation sugar is a monosaccharide or a disaccharide. In preferred embodiments, the sugar is a mixture of sucrose and trehalose, each present in a concentration ranging from about 5% to about 40% (w/v).

Divalent cation embodiments are provided. Certain divalent cations can help stabilize viral membrane structures. Divalent cations, in a pharmaceutically acceptable salt form, can also be useful in modifying the osmolarity of the virus vaccine-containing solution. In some embodiments of the invention, cations are present in the formulation in amounts ranging from about 0.1 mM to about 100 mM. In preferred embodiments, one or more divalent cations are present ranging in concentration from about 1 mM to about 20 mM, or from about 2 mM to about 10 mM.

Preferred divalent cations for incorporation into the inventive formulations are, e.g., pharmaceutically acceptable salts of magnesium, zinc, calcium, manganese, and their combinations thereof. In a most preferred embodiment, a mixture of calcium and zinc, both as a chloride salt, is incorporated into said formulation, e.g., at a concentration ranging from about 2 mM to about 10 mM for each divalent cation.

Amino acid embodiments are encompassed. Amino acids can help stabilize viral membrane structures and contribute to pH buffering. Amino acids can also be useful in modifying the osmolarity of the virus vaccine-containing solution, the solution pH, and the surface tension of solution during processing, e.g. foam drying and spray drying. In some embodiments of the invention, amino acids are present in the formulation in amounts ranging from about 0.1% to 10% (w/v). In preferred embodiments, one or more amino acids are present at a concentration ranging from about 1% to about 8% (w/v), or about 4% (w/v).

Preferred amino acids for incorporation into the inventive formulations are, e.g., alanine, arginine, methionine, serine, lysine, histidine, glutamic acid, and/or the like. In a most preferred embodiment, the amino acid is arginine, e.g., at a concentration ranging from about 1% to about 8% (w/v).

Polymer embodiments are provided. Formulations of the present invention appear to benefit from the presence of a polymer in the formulation. Similar to polyols, polymers can be included to increase the solution viscosity and to provide structural strength during a drying process, e.g. foam drying and freeze drying. In case of spray drying, polymers can be included to modify the surface properties of atomized droplets. In preferred embodiments, the polymer is ingestible. Preferably, the polymer has significant ionic character, preferably anionic character. In certain embodiments, the polymer is present in a concentration ranging from about 0.1% to 20% (w/v), or about 4% (w/v).

More preferred polyols include, e.g., gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, human serum albumin, lactalbumin hydrolysate, and their combinations thereof. In one embodiment, the polymer is human serum albumin. In certain embodiments, the formulation comprises from about 1% to about 10% (w/v) human serum albumin.

Plasticizer embodiments are embraced, by the invention. Formulations of the present invention appear to benefit from the presence of a plasticizer in the formulation. In some embodiments of the invention, plasticizers are present in the formulation in amounts ranging from about 0.1% to about 5% by weight of said formulation. In preferred embodiments, one or more plasticizers are present at a concentration ranging from about 0.1% to about 5%, or about 1% by weight of said formulation.

More preferred plasticizers include, e.g., glycerol, dimethylsulfoxide (DMSO), propylene glycol, ethylene glycol, oligomeric polyethylene glycol, sorbitol, and their combinations thereof. In one embodiment, the plasticizer is glycerol. In certain embodiments, the formulation comprises from about 0.5% to about 3% glycerol by weight of said formulation.

Surfactants are encompassed. Formulations of the present invention appear to benefit from the presence of a surfactant in the formulation. Furthermore, the surfactant may be included to aid in certain drying processes, e.g. foam drying and spray drying, by decreasing the surface tension and in coating the particle surface, respectively. Surfactants may also be included to enhance the solubility of other formulation constituents. In certain embodiments, the surfactant is present in a concentration ranging from about 0.01% to about 2% by weight of said formulation, or about 0.1%. In a preferred embodiment, the formulation does not contain any surfactant.

More preferred surfactants include, e.g., polyethylene glycol, polypropylene glycol, polyethylene glycol/polypropylene glycol block copolymers, polyethylene glycol alkyl ethers, polyethylene glycol sorbitan monolaurate, polypropylene glycol alkyl ethers, polyethylene glycol/polypropylene glycol ether block copolymers, polyoxyethylenesorbitan monooleate, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde and phenol, lignin-sulfite waste liquor, alkyl phosphates, quaternary ammonium compounds, amine, oxides, betaines, and/or the like. In one embodiment, the surfactant is block copolymers of polyethylene and polypropylene glycol. In certain embodiments, the formulation comprises from about 0.01% to about 0.1% block copolymers of polyethylene and polypropylene glycol by weight of said formulation.

Buffers are provided by the invention. Pharmaceutically acceptable buffering components are included in the present invention to adjust the pH and the osmolarity of the formulation. In some embodiments of the invention, buffers are present in the formulation in concentration ranging from about 5 mM to about 1M. In preferred embodiments, one or more buffering components are present at a concentration ranging from about 5 mM to about 200 mM, or from about 50 mM to about 100 mM.

Preferred buffers for incorporation into the inventive formulations are, e.g., potassium phosphate, sodium phosphate, sodium acetate, histidine, imidazole, sodium citrate, sodium succinate, ammonium bicarbonate, a carbonate, and/or the like. In a most preferred embodiment, the buffer is potassium phosphate ranging in concentration from about 50 mM to about 100 mM.

Typically, the pH of the inventive formulation is adjusted to provide a physiological pH, such as pH7.4, a pH ranging from about pH 4 to about pH9, from pH 5 to pH 8, or about pH 7. Buffering capacity of the current invention can be provided by the buffer or an amino acid, if included.

Preferred combinations of constituents are disclosed. Preferred combinations of pharmaceutically acceptable excipient constituents that enhance the stability of spray dried, freeze dried, and foam dried formulations of measles virus may include, e.g., combinations of a sugar and potassium phosphate buffer ranging in concentration from about 50 mM to about 200 mM adjusted to a pH of about 6 to 7. In more preferred embodiments, the sugar can be a mixture of sucrose and trehalose, each present at a concentration ranging from about 5% to about 20% (w/v).

Furthermore, it can be beneficial to include a pharmaceutically acceptable salt form of certain divalent cations, such as CaCl₂ and ZnCl₂, or their mixtures thereof, e.g., at a concentration ranging from about 1 mM to about 20 mM, or more preferably from about 2 mM to about 10 mM for each divalent cation.

In addition to the above combinations of constituents, it can be beneficial to include an amino acid, such as arginine, e.g., at a concentration ranging from about 1% to about 8% (w/v), or about 4% (w/v).

Furthermore, it can be beneficial to include a polymer to the combination of constituents described above, such as human serum albumin, e.g., at a concentration ranging from about 1% to about 10% (w/v). In more preferred embodiments, the polymer can be human serum albumin at a concentration of about 4% (w/v).

In addition to the combinations of constituents described above, it can be beneficial to include a plasticizer, such as glycerol, e.g., at a concentration ranging from about 0.5% to about 3% by weight of said formulation.

In the present inventive formulations, the presence of surfactants can possibly enhance stability. Furthermore, the choice of processing method to prepare dry measles vaccine, e.g., foam drying and spray drying, may dictate the use of surfactants. In more preferred embodiments, the surfactant can be block copolymers of polyethylene and polypropylene glycol at a concentration ranging from about 0.01% to 0.1% by weight of said formulation.

Dry powder production is included in the present invention. In another aspect of the present invention, the virus vaccine-containing compositions may be prepared as a dry powder. Dry powder production can be conducted employing a variety of methods known to those skilled in the art, which includes, but is not limited to spray drying, freeze drying, foam drying, spray freeze drying, fluidized bed drying, supercritical fluid assisted drying, and vacuum drying. Spray drying is most preferable for use in the present invention.

In an exemplary embodiment of the inventive methods, a solution containing measles virus is first formulated with stabilizing excipients, as described above, then atomized from a nozzle using a pressurized gas, with or without an organic solvent serving as a liquid modifier. The atomized droplets are caused to dry into powder particles by infusing a stream of dry, heated gas co-current to the spray plume. The spray drying equipment can be any commercially available spray dryers fitted with any commercially available atomizing nozzles. The atomizing gas can be air or any other gases, preferably air, nitrogen, CO₂ at or near supercritical state. The gas used to evaporate the atomized solution, i.e. the drying gas, is typically heated and can be air, nitrogen, argon, or the like.

Droplets of suspensions or solutions can be dried to form particles. The drying can be conducted by any means appropriate to the droplet composition and intended use. For example, the droplets can be sprayed into a stream of drying gas, onto a drying surface, into a cold fluid to freeze the droplets for later lyophilization, and/or the like. Dry particles are typically not liquid and can have moisture content (e.g., residual moisture) of less than 15%, less than 10%, less than 5%, less than 3%, less than 1.5% or about 1%.

In one embodiment, the droplets are sprayed into a stream of a drying gas. For example, the drying gas can be an inert gas, such as nitrogen, at a temperature ranging from ambient temperatures to 200° C. In many cases, the stream of drying gas can enter the drying chamber to contact the droplets at a temperature of 150° C. or less, 100° C., 70° C., 50° C., 30° C. or less. The particles can be collected by settling, filtration, impact, etc. Particles can be exposed to secondary drying conditions to remove additional moisture.

The dry powder particles produced by spray drying can be room temperature stable and exhibit appropriate powder properties for deep lung delivery as well as for fabrication into other dosage formats such as oral wafers, oral thin films, capsules, tablets, etc.

Alternatively, the droplets can be lyophilized to dryness. The freeze drying equipment can be any commercially available freeze dryers. A solution of measles virus is first formulated with stabilizing excipients, and then frozen. The freezing step can be done either slowly, e.g. by cooling on the shelf of the freeze dryer, or quickly, e.g. by quenching in liquid nitrogen. Preferably, the freezing is done slowly on a pre-cooled shelf set at −50° C. Other freezing methods may also be employed to freeze the measles virus solution prior to the primary drying cycle. The freeze drying process is comprised of primary drying and secondary drying cycles. The primary drying cycle is conducted at or below −20° C., more preferably at −35° C., under vacuum of at least 100 mTorr, preferably at or below 50 mTorr. The duration of the cycle may be up to 4000 min. Prior to secondary drying, there may be an additional step, in which the temperature of the shelf may be raised to 0° C. under 50 mTorr. This step may last up to 1000 min, more preferably under 500 min. The secondary drying is generally conducted above 10° C., preferably above 20° C., and more preferably at or above 28° C., under vacuum of at least 100 mTorr, preferably at or below 50 mTorr. The secondary drying step may last up to 2000 min, preferably under 1000 min. In another embodiment, the droplets are sprayed into liquid nitrogen to form frozen droplets.

In another embodiment, the measles virus-containing solutions can be foam dried. Foam drying methods generally consist of, e.g., processes of expanding a formulation of measles virus into a foam, followed by drying the foam into a stable dry foam composition. The methods can variously include, e.g., freezing of the foam before drying, inclusion of foaming agents in the formulation, holding the formulation at the phase transition temperature of a lipid membrane to enhance penetration of protective agents, expansion of the formulation at pressures between about 200 Torr and 25 mTorr and/or secondary drying to further reduce the moisture content. For a detailed description of the process, see U.S. Pat. No. 7,371,425 “Preservation of bioactive materials by freeze dried foam”.

Buffer embodiments that are encompassed by the invention, include a composition or formulation with less than about 100 mM potassium, less than 75 mM K⁺, less than 50 mM K⁺, less than 40 mM K⁺, less than 30 mM K⁺, less than 20 mM K⁺, less than 10 mM K⁺, less than 5 mM K⁺, or with no K⁺. In another aspect, what is encompassed is a composition or formulation with about 10 mM sodium, about 20 mM Na⁺, about 30 mM Na⁺, about 40 mM Na⁻, about 50 mM Na⁺, about 60 mM Na⁺, about 70 mM Na⁺, about 80 mM Na⁺, about 90 mM Na⁺, about 100 mM Na⁺, and the like. Moreover, what is encompassed is a composition or formulation with about 50 mM sodium, and less than 50 mM potassium, less than 40 mM K⁺, less than 30 mM K⁺, less than 20 mM K⁺, less than 10 mM K⁺, less than 5 mM K⁺, and the like. Also provided is a composition or formulation with about 25 mM sodium, and less than 50 mM potassium, less than 40 mM K⁺, less than 30 mM K⁺, less than 20 mM K⁺, less than 10 mM K⁺, less than 5 mM K⁺, or with no potassium.

In another aspect, what is provided is a composition or formulation, that includes excludes a stability-degrading concentration of potassium, where this concentration is that where stability in the presence of a given molarity (M) of potassium ions is inferior to the stability where the same concentration (M) of sodium ions replaces the potassium ions. In one embodiment, the stability is process stability, while in another embodiment, the stability is storage stability, and also, the stability can be a composite of process and storage stability. In yet another aspect, the composition or formulation excludes a stability-degrading concentration of potassium, and includes no sodium, 0-5 mM sodium, 5-10 mM sodium, 10-20 mM sodium, 20-30 mM sodium, 30-40 mM sodium, 40-50 mM sodium, 50-60 mM sodium, 60-70 mM sodium, 70-90 mM sodium, 90-100 mM sodium, and the like.

In divalent cation embodiments, the present invention provide a composition or formulation with about 1 mM calcium and zinc at about 0 mM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 30 mM, 40 mM, 50 mM, and the like. In another divalent cation embodiment what is provided is a composition or formulation with about 5 mM calcium, and zinc at about 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, and the like. In the composition and formulation embodiment, what is also encompassed is about 10 mM calcium, and zinc at about 0 mM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 30 mM, 40 mM, 50 mM, and the like. Moreover, what is provided in the composition and formulation embodiments, is about 20 mM calcium, and zinc at about 0 mM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 30 mM, 40 mM, 50 mM, and the like. In alternate embodiments, calcium can be at about 0 mM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 30 mM, 40 mM, 50 mM, and the like. In certain embodiments, the calcium must be under about 100 mM, under about 80 mM, under about 60 mM, under about 50 mM, under about 40 mM, under about 30 mM, under about 20 mM, or under about 10 mM, while in certain other embodiments, the zinc must be under about 100 mM, under about 80 mM, under about 60 mM, under about 50 mM, under about 40 mM, under about 30 mM, under about 20 mM, or under about 10 mM.

Stability-enhancing concentrations of divalent cation, calcium only, calcium in the absence of zinc, zinc only, zinc in the absence of calcium, and combinations of calcium and zinc, are provided. In one embodiment, the stability is process stability, while in another embodiment, the stability is storage stability, and also, the stability can be a composite of process and storage stability. In a stability-enhancing concentration of divalent cation, the stability is at least 1.5-fold, 2-fold, 3-fold, 4-fold, 5-fold, 6-fold, 7-fold, 8-fold, 10-fold, 12-fold, 14-fold, 16-fold, 18-fold, 20-fold, or more, times stable, than in the absence of that divalent cation.

In another aspect, the invention comprises a vaccine that is made using a formulation, or that is prepared with a formulation, where the formulation has greater than 10% solids, greater than 12% solids, greater than 15% solids, greater than 18% solids, greater than 20% solids, greater than 25% solids, greater than 30% solids, and the like.

In another aspect, the invention comprises a vaccine that is made using a formulation, or that is prepared with a formulation, where the formulation has greater than 10% solids and less than 40% solids, greater than 12% solids and less than 40% solids, greater than 15% solids and less than 40% solids, greater than 18% solids and less than 40% solids, greater than 20% solids and less than 40% solids, greater than 25% solids and less than 40% solids, greater than 30% solids and less than 40% solids, and the like.

In other embodiments, what is provided is a vaccine made using a formulation, where the formulation has less than 50% solids, less than 45% solids, less than 40% solids, less than 35% solids, less than 30% solids, less than 25% solids, and so on.

The present invention, is made from a formulation that does not have a buffer, or that has low concentrations of a buffer that avoid crystallization that might occur during drying of the combination of virus plus formulation, or that contains a buffer that does not crystallize during drying of the combination of virus plus formulation, or that contains a buffer plus an additive where the additive prevents the buffer from crystallizing during drying of the formulation. Crystals have the potential to damage the virus that is in the present vaccine. What is also contemplated is a formulation that contain a salt that is not a buffer, and where the salt in the formulation is at a limited concentration, where this limited concentration avoids crystallization of the salt during drying of the combination of the vaccine and formulation.

### EXAMPLES

The following examples are offered to illustrate, but not to limit the scope of the claimed invention.

### Example 1

### Effect of Spray Drying Process Conditions on The Recovery of Measles Infectivity

Measles virus was spray dried using an ultrasonic nozzle at low pressure under the conditions shown in Table 2. Process-associated loss, as well as the loss in virus titer after 1 week of storage at 37° C., residual moisture content, and glass transition temperature (T_{g}) are also shown in Table 2. Virus infectivity was measured by tissue culture infectivity dose (TCID) assay. In this example, and those that follow, the strain of measles was the Edmonston-Zagreb strain.

TABLE 2
Spray Drying Process Parameters, Process Recovery,
and Storage Stability of Measles Virus.

| | | | Storage loss | | |
|---|---|---|---|---|---|
| | | Process loss | 1 week, 37° C. | Residual | T_{g} |
| Process | Parameters | (Log TCID₅₀) | (Log TCID₅₀) | moisture (%) | (° C.) |
| A | Pₐₜₘ = 24 psi | 0.3 | 1.8 | 1.4 | 50-60 |
| | q = 0.5 mL/min | | | | |
| | Tₒᵤₜ = 60° C. | | | | |
| B | Pₐₜₘ = 15 psi | 0.2 | 1.4 | 2.3 | 50-60 |
| | q = 0.5 mL/min | | | | |
| | Tₒᵤₜ = 60° C. | | | | |
| C | Pₐₜₘ = 15 psi | 0.0 | 1.5 | 4.3 | 50-60 |
| | q = 1 mL/min | | | | |
| | Tₒᵤₜ = 40° C. | | | | |
| D | Pₐₜₘ = 15 psi | 0.5 | 0.8 | 3.6 | 53 |
| | q = 0.5 mL/min | | | | |
| | Tₒᵤₜ = 40° C. | | | | |

### Example 2

### Effect of Measles Virus Titer on Process Recovery

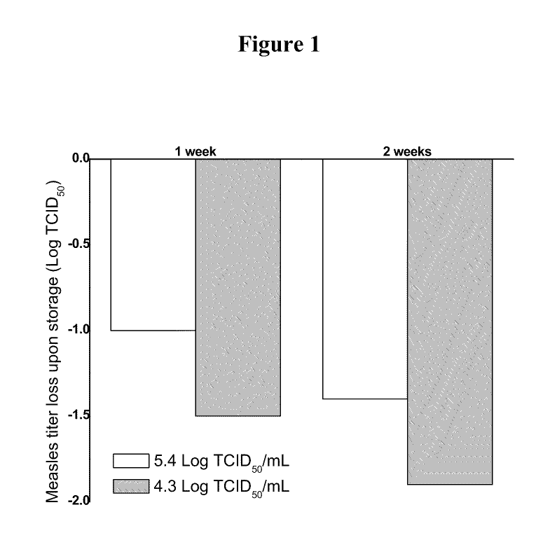

Measles virus was spray dried using an ultrasonic nozzle at low pressure under the following conditions:
a) liquid formulations of measles virus titrated at either 4.3 or at 5.4 Log TCID₅₀/mL containing 8.3% (w/v) trehalose, 12.7% (w/v) sucrose, 4% (w/v) L-arginine, 1.25% (wt) glycerol, and 0.06% (wt) Pluronic F68 in 69.4mM potassium phosphate buffer adjusted to pH7;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C. (i.e. outlet temperature);
e) dry powder was collected and reconstituted to determine the process-associated loss; losses in titer of 0.1 and 0.4 Log TCID₅₀ were observed for samples with initial virus titer of 5.4 and 4.3 Log TCID₅₀/mL, respectively.
f) the dry powder was placed in glass vials, capped, and sealed. The vials were stored at 37° C. and taken out at various time points to determine viral infectivity (FIG. 1).

### Example 3

### Effect of Buffer Concentration on the Storage Stability of Spray Dried Measles Virus

Measles virus was spray dried using an ultrasonic nozzle at low pressure under the following conditions:
a) liquid formulations of measles virus were titrated to about 4.3 Log TCID₅₀/mL using formulation components listed in Table 3;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder containing less than 3% residual moisture content was collected. Viral particle concentration following reconstitution is shown in Table 4.
f) the dry powder was placed in glass vials, capped, and sealed. The vials were stored at 37° C. and taken out at various time points to determine the viral infectivity (Table 4).

TABLE 3
Formulation Composition for Spray Dried Measles Virus

| | Formulation | | | | |
|---|---|---|---|---|---|
| Components | M1 | M2 | M3 | M4 | M5 |
| Trehalose (%, w/v) | 8.3 | 8.3 | 8.3 | 8.3 | 8.3 |
| Sucrose (%, w/v) | 12.7 | 12.7 | 12.7 | 12.7 | 12.7 |
| KPO₄ (mM) | | 25 | 50 | 70 | |
| NaPO₄ (mM) | | | | | 50 |
| L-arginine (%, w/v) | 4 | 4 | 4 | 4 | 4 |
| Glycerol (%, wt) | 1.25 | 1.25 | 1.25 | 1.25 | 1.25 |
| Pluronic F68 (%, wt) | 0.06 | 0.06 | 0.06 | 0.06 | 0.06 |
| pH | 7 | 7 | 7 | 7 | 7 |

TABLE 4
Viral titer of initial solution, immediately
after spray drying and upon storage at 37° C.

| | Log TCID₅₀/mL | | | |
|---|---|---|---|---|
| Formulation | Solution | Post-spray drying | 1 week | 2 weeks |
| M1 | 4.3 | 4.0 ± 0.0 | 2.6 ± 0.1 | 2.7 ± 0.1 |
| M2 | 4.1 | 4.2 ± 0.1 | 3.1 ± 0.1 | 2.7 ± 0.2 |
| M3 | 4.5 | 4.4 ± 0.1 | 3.0 ± 0.2 | 2.7 ± 0.5 |
| M4 | 4.4 | 3.9 ± 0.1 | 2.4 ± 0.6 | 2.0 ± 0.1 |
| M5 | 4.4 | 4.4 ± 0.1 | 3.0 ± 0.2 | 2.9 ± 0.1 |

### Example 4

### Effect of Divalent Cations on the Storage Stability of Spray Dried Measles Virus

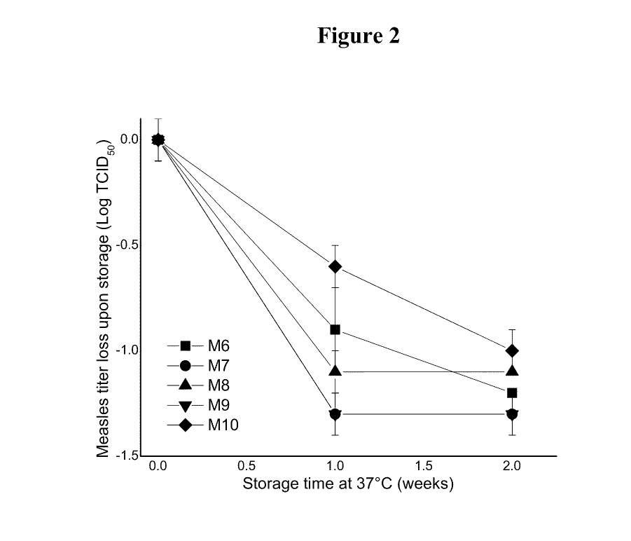

Measles virus was spray dried using an ultrasonic nozzle at low pressure under the following conditions:
a) liquid formulations of measles virus were titrated to about 4.3 Log TCID₅₀/mL using formulation components listed in Table 5;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder was collected and reconstituted to determine the process-associated loss in virus titer (Table 5).
f) the dry powder was placed in glass vials, capped, and sealed. The vials were stored at 37° C. and taken out at various time points to determine the viral infectivity (FIG. 2).

TABLE 5
Formulation Composition for Spray Dried Measles Virus

| | Formulation | | | | |
|---|---|---|---|---|---|
| Components | M6 | M7 | M8 | M9 | M10 |
| Trehalose (%, w/v) | 8.3 | 8.3 | 8.3 | 8.3 | 8.3 |
| Sucrose (%, w/v) | 12.7 | 12.7 | 12.7 | 12.7 | 12.7 |
| KPO₄ (mM) | 69.4 | 69.4 | 69.4 | 69.4 | 69.4 |
| L-arginine (%, w/v) | 4 | 4 | 4 | 4 | 4 |
| Glycerol (%, wt) | 1.25 | 1.25 | 1.25 | 1.25 | 1.25 |
| Pluronic F68 (%, wt) | 0.06 | 0.06 | 0.06 | 0.06 | 0.06 |
| MgCl₂ (mM) | | 2 | | | |
| CaCl₂ (mM) | | | 2 | | 2 |
| ZnCl₂ (mM) | | | | 2 | 2 |
| pH | 6 | 6 | 6 | 6 | 6 |
| Process loss (Log TCID₅₀) | 0.6 | 0.5 | 0.5 | 0.2 | 0 |

### Example 5

### Effect of Formulation pH on the Storage Stability of Spray Dried Measles Virus

Measles virus was spray dried using an ultrasonic nozzle at low pressure under the following conditions:
a) liquid formulations of measles virus were titrated to about 4.3 Log TCID₅₀/mL using formulation components listed in Table 6;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder was collected and reconstituted to determine the process-associated loss in virus titer (Table 7).
f) the dry powder was placed in glass vials, capped, and sealed. The vials were stored at 37° C. and taken out at various time points to determine the viral infectivity (Table 7).

TABLE 6
Formulation Composition for Spray Dried Measles Virus

| Components | M6 | M4 | M10 | M11 | M12 |
|---|---|---|---|---|---|
| Trehalose (%, w/v) | 8.3 | 8.3 | 8.3 | 8.3 | 8.3 |
| Sucrose (%, w/v) | 12.7 | 12.7 | 12.7 | 12.7 | 12.7 |
| KPO₄ (mM) | 69.4 | 69.4 | 69.4 | 69.4 | 69.4 |
| L-arginine (%, w/v) | 4 | 4 | 4 | 4 | 4 |
| Glycerol (%, wt) | 1.25 | 1.25 | 1.25 | 1.25 | 1.25 |
| Pluronic F68 (%, wt) | 0.06 | 0.06 | 0.06 | 0.06 | 0.06 |
| CaCl₂ (mM) | | | 2 | 2 | 2 |
| ZnCl₂ (mM) | | | 2 | 2 | 2 |
| pH | 6 | 7 | 6 | 7 | 8 |

TABLE 7
Viral titer of initial solution, immediately
after spray drying and upon storage at 37° C.

| | Log TCID₅₀/mL | | | |
|---|---|---|---|---|
| Formulation | Solution | Post-spray drying | 1 week | 2 weeks |
| M4 | 4.3 | 3.9 | 2.4 | 2.0 |
| M6 | 4.3 | 3.7 ± 0.1 | 2.8 ± 0.2 | 2.5 ± 0.0 |
| M10 | 3.9 | 3.9 ± 0.1 | 3.3 ± 0.1 | 2.9 ± 0.1 |
| M11 | 4.3 | 4.3 ± 0.2 | 3.1 ± 0.9 | 3.3 ± 0.1 |
| M12 | 4.6 | 4.4 ± 0.1 | 3.3 ± 0.3 | 2.6 ± 0.3 |

### Example 6

### Effect of Proteins on the Storage Stability of Spray Dried Measles Virus

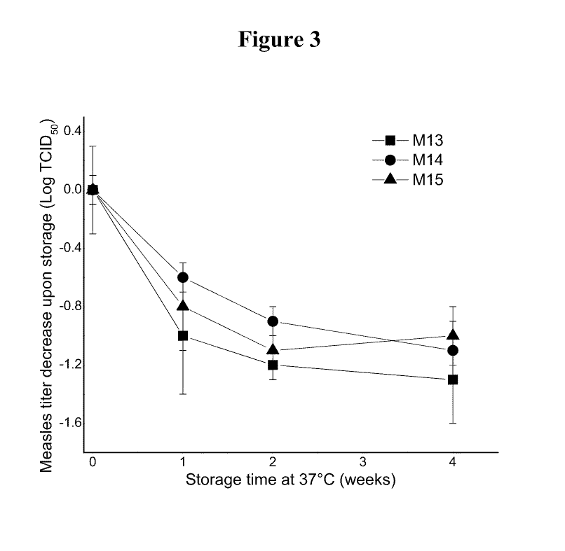

Measles virus was spray dried using an ultrasonic nozzle at low pressure under the following conditions:
a) liquid formulations of measles virus were titrated to about 4.3 Log TCID₅₀/mL using formulation components listed in Table 8;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder was collected and reconstituted to determine the process-associated loss in virus titer. None of the formulations demonstrated any decrease in titer upon spray drying.
f) the dry powder was placed in glass vials, capped, and sealed. The vials were stored at 37° C. and taken out at various time points to determine the viral infectivity (FIG. 3)

TABLE 8
Formulation Composition for Spray Dried Measles Virus

| Components | M13 | M14 | M15 |
|---|---|---|---|
| Trehalose (%, w/v) | 7.3 | 7.3 | 6.3 |
| Sucrose (%, w/v) | 11.7 | 11.7 | 10.7 |
| KPO₄ (mM) | 69.4 | 69.4 | 69.4 |
| L-arginine (%, w/v) | 4 | 4 | 4 |
| Glycerol (%, wt) | 1.25 | 1.25 | 1.25 |
| Pluronic F68 (%, wt) | 0.06 | 0.06 | 0.06 |
| Gelatin (%, w/v) | 2 | | |
| Human Serum Albumin (%, w/v) | | 2 | 4 |
| CaCl₂ (mM) | 2 | 2 | 2 |
| ZnCl₂ (mM) | 2 | 2 | 2 |
| pH | 7 | 7 | 7 |

For this table, a comparable control that lacks protein can be found in Table 6 (M11).

### Example 7

### Effect of Surfactants on the Storage Stability of Spray Dried Measles Virus

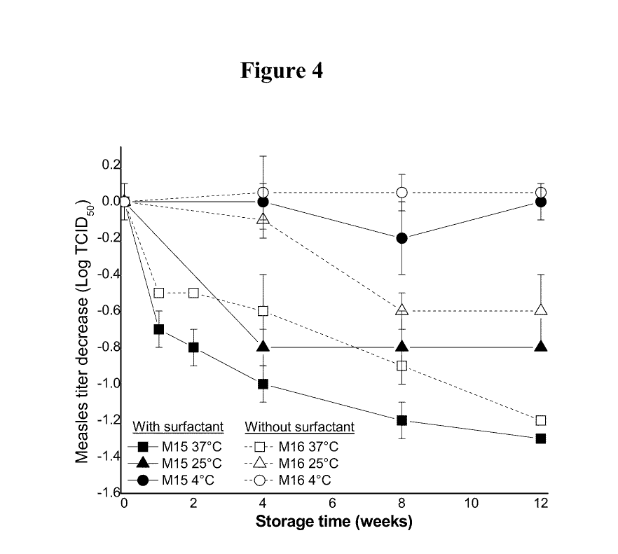

Measles virus was spray dried using an ultrasonic nozzle at low pressure under the following conditions:
a) liquid formulations of measles virus were titrated to about 4.3 Log TCID₅₀/mL using formulation components listed in Table 9;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder was collected and reconstituted to determine the process-associated loss in virus titer (Table 10).
f) the dry powder was placed in glass vials, capped, and sealed. The vials were stored at 4° C., 25° C., and 37° C. and taken out at various time points to determine the viral infectivity (FIG. 4).

TABLE 9
Formulation Composition for Spray Dried Measles Virus

| Components | M16 | M15 |
|---|---|---|
| Trehalose (%, w/v) | 6.3 | 8.3 |
| Sucrose (%, w/v) | 10.7 | 12.7 |
| KPO₄ (mM) | 69.4 | 69.4 |
| L-arginine (%, w/v) | 4 | 4 |
| Glycerol (%, wt) | 1.25 | 1.25 |
| Pluronic F68 (%, wt) | 0 | 0.06 |
| Human Serum Albumin (%, w/v) | 4 | 4 |
| CaCl₂ (mM) | 2 | 2 |
| ZnCl₂ (mM) | 2 | 2 |
| pH | 7 | 7 |

TABLE 10
Viral titer of initial solution and of post-spray drying

Log TCID₅₀/mL

| Formulation | Solution | Post-spray drying |
|---|---|---|
| M16 | 4.4 | 3.9 ± 0.0 |
| M15 | 4.3 | 4.1 ± 0.3 |

The following concerns surfactants. With regards to the surfactant, the presence of another surface active molecule, such as human serum albumin, the two components may compete against each other and result in destabilized stability. Protein appears to be more stabilizing than surfactant alone, if only one of the two components are to be used.

### Example 8

### Effect of Solids Content on the Storage Stability of Spray Dried Measles Virus

Measles virus was spray dried using an ultrasonic nozzle at low pressure under the following conditions:
a) liquid formulations of measles virus were titrated to about 4.3 Log TCID₅₀/mL using formulation components listed in Table 11;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C.;
e) dry powder was collected and reconstituted to determine the process-associated loss in virus titer (Table 12).
f) the dry powder was placed in glass vials, capped, and sealed. The vials were stored at 37° C. and taken out at various time points to determine the viral infectivity (Table 12).

TABLE 11
Formulation Composition for Spray Dried Measles Virus

| Components | M17 | M14 | M18 |
|---|---|---|---|
| Trehalose (%, w/v) | 2.94 | 7.3 | 11.8 |
| Sucrose (%, w/v) | 4.67 | 11.7 | 18.8 |
| KPO₄ (mM) | 50 | 50 | 50 |
| L-arginine (%, w/v) | 1.6 | 4 | 6.4 |
| Human Serum Albumin (%, w/v) | 0.8 | 2 | 3.2 |
| Glycerol (%, wt) | 1.25 | 1.25 | 1.25 |
| Pluronic F68 (%, wt) | 0.06 | 0.06 | 0.06 |
| CaCl₂ (mM) | 2 | 2 | 2 |
| ZnCl₂ (mM) | 2 | 2 | 2 |
| pH | 7 | 7 | 7 |
| Solids content (%, w/v) | 10 | 25 | 40 |

TABLE 12
Viral titer of initial solution, immediately
after spray drying and upon storage at 37° C.

| | Log TCID₅₀/mL | | | | |
|---|---|---|---|---|---|
| | | Post-spray | | | |
| Formulation | Solution | drying | 1 week | 2 weeks | 4 weeks |
| M17 | 4.1 | 4.1 ± 0.1 | 2.8 ± 0.1 | 2.7 ± 0.1 | 2.6 ± 0.1 |
| M14 | 4.2 | 4.4 ± 0.1 | 3.8 ± 0.1 | 3.5 ± 0.1 | 3.3 ± 0.1 |
| M18 | 4.1 | 4.0 ± 0.1 | 3.2 ± 0.2 | 2.8 ± 0.1 | 2.5 ± 0.1 |

### Example 9

### Effect of Processing Method on the Storage Stability of Dry Measles Virus

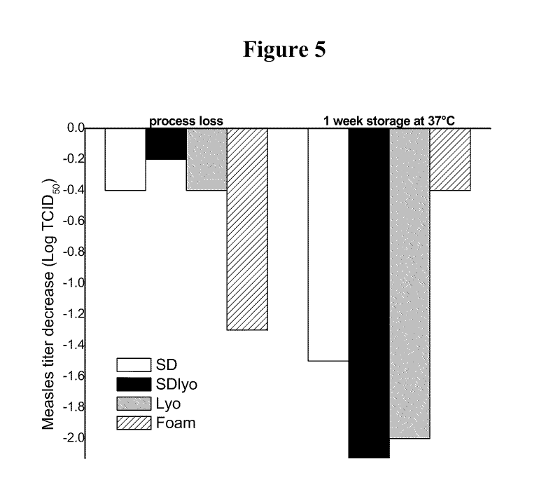

Dry measles virus was prepared by a variety of processing methods, including spray drying, with and without secondary drying, freeze drying, and foam drying. The measles virus was titrated to 4.3 Log TCID₅₀/mL, employing a formulation containing 8.3% (w/v) trehalose, 12.7% (w/v) sucrose, 4% (w/v) L-arginine, 1.25% (wt) glycerol, and 0.06% Pluronic F68 in 69.4 mM potassium phosphate buffer adjusted to pH7. Spray drying was conducted using the procedure described above in Example 8. In addition, some of the spray dried samples were further processed by employing a secondary drying phase to reduce the residual moisture content. Secondary drying was conducted at 50 mTorr and 15° C. for about 12 hours. For freeze drying, the formulated measles virus was: 1) placed on a pre-cooled shelf set at −50° C. and frozen for 140 min, 2) dried at −35° C. and 50 mTorr for 3850 min followed by 0° C. and 50 mTor for 485 min (primary drying), and 3) dried further at 28° C. and 50 mTorr for 1020 min (secondary drying). For foam drying, the formulated measles virus was processed according to: 1) 15° C. at atmospheric pressure for 10 min, 2) 15° C. at or below 50 mTorr for 24 hours, and 3) 33° C. at or below 50 mTorr for 24 hours. The process-associated loss in measles titer and the measles titer loss after 1 week storage at 37° C. are shown in FIG. 5.

### Example 10

### Effect of Formulation Composition on the Process Recovery of Adenovirus

Adenovirus, serotype Ad4, was spray dried using an ultrasonic nozzle at low pressure under the following conditions:
a) liquid formulations of adenovirus titrated at about 1×10¹¹ viral particles/mL containing the components listed in Table 13 were prepared;
b) the formulation, at a flow rate of 0.5 mL/min, was combined with a stream of nitrogen gas at 15 psi in the mixing chamber of the nozzle;
c) the nozzle was vibrated at ultrasonic frequencies;
d) the formulation/gas mixture was sprayed into a drying chamber while the drying gas flowed into the chamber at 60° C. Drying gas exited the chamber at 40° C. (i.e. outlet temperature);
e) dry powder containing less than 3% residual moisture content was collected. Viral particle concentration following reconstitution is shown in Table 13.
f) process-recovery of viral infectivity, or viral structural integrity, was assessed by anion-exchange chromatography. Briefly, a Resource Q™ column was used with mobile phase consisting of 50 mM Tris at pH7.5, with the elution of viral particles controlled by NaCl concentration gradient. The analytical method employed is not limited to the one chosen here, but other methods may be equally applicable to determine the relative decrease in viral infectivity.

TABLE 13
Formulation Composition for Spray Dried Adenovirus

| Components | A1 | A2 | A3 | A4 | A5 | A6 | A7 | A8 | A9 |
|---|---|---|---|---|---|---|---|---|---|
| Sucrose (%, w/v) | 20 | 20 | 20 | 20 | 20 | 20 | 20 | | |
| Trehalose (%, w/v) | | | | | | | | 20 | 15 |
| KPO₄ (mM) | 50 | 50 | 50 | 100 | 200 | 200 | 200 | 50 | 50 |
| Glycerol (%, wt) | | 2 | 2 | 2 | 2 | 2 | 2 | | 2 |
| Pluronic F68 (%, wt) | | 0.04 | 0.04 | 0.04 | 0.04 | 0.04 | 0.04 | | 0.04 |
| MgCl₂ (mM) | | | 2 | 2 | 2 | 5 | 10 | | 2 |
| Gelatin (%, w/v) | | | | | | | | | 5 |
| pH | 7 | 7 | 7 | 7 | 7 | 7 | 7 | 7.4 | 7.4 |
| Process loss (Log₁₀₎ | 1.38 | 0.79 | 0.54 | 0.58 | 0.29 | 0.27 | 0.0 | 1.87 | 0.96 |

### Example 11

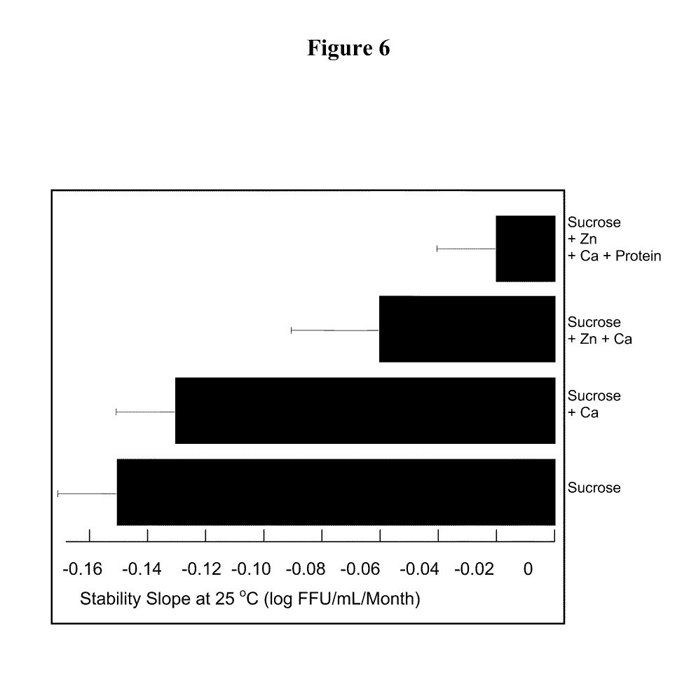

FIG. 6 shows the stabilization of antacid-containing monovalent bovine rotavirus (BRV) vaccine, strain G3, processed by spray drying. Spray drying was conducted using a Buchi 190 mini-spray dryer equipped with an ultrasonic nozzle, at low pressure, and typically under the condition of: 0.5 mL/min solution feed rate, 60° C. inlet temperature, and 45° C. outlet temperature. Rotavirus was mixed with citrate/phosphate-based antacid (0.8 mEq), buffered at approximately pH 6.3, following incorporation of formulation components, including sucrose with and without calcium, zinc, and a protein. Titer of rotavirus was measured using fluorescent focus assay (FFA), and the pre-spray drying value was determined to be approximately 5.9 log₁₀ ffu/mL. In various formulations, sucrose ranged from 5 to 40% (w/v), zinc and calcium were typically present at 2 mM, but were examined up to 10 mM, and the protein was gelatin, typically less than 50 wt % of the sugar concentration. Rotavirus vaccine was processed by both freeze drying and spray drying. The stability slope shown in the figure was obtained following prolonged storage of the spray dried rotavirus vaccine at 25° C.

### Example 12

### Effect of Formulation Composition on the Process Recovery of Live Attenuated Salmonella Typhi Bacterial Vaccine

Live attenuated Salmonella typhi vaccine, Ty21a, was cultured by inoculation in brain heart infusion (BHI) broth overnight at 37° C. and harvested in the early stationary phase (˜2.2 OD_{600 nm}). The sample was centrifuged at 2500 rcf for 10 minutes, and the resulting bacterial pellet was resuspended in the formulations shown below (Table 14), and taken to the initial volume. The resulting solutions were spray dried using a Buchi 190 mini-spray dryer equipped with an ultrasonic nozzle, at low pressure, and under the condition of: 0.75 mL/min solution feed rate, 45° C. inlet temperature, and 35° C. outlet temperature. The recovered powder was reconstituted with double-filtered deionized water and plated out on TSB plates for viability determination. The plates were counted after 16 hours of incubation at 37° C. The process-associated loss for the examined formulation is shown in Table 14.

TABLE 14
Formulation composition and process-
associated loss for spray dried Ty21a

| Components | T1 | T2 | T3 | T4 |
|---|---|---|---|---|
| Sucrose (%, w/v) | 7 | | 7 | 7 |
| Trehalose (%, w/v) | | | | 3 |
| Leucine (%, w/v) | | 2 | 2 | |
| Pluronic F68 (wt %) | | | | 0.02 |
| Glycerol (wt %) | | | | 0.25 |
| KPO₄ (mM) | | | | 25 |
| Process loss (Log₁₀) | 0.9 | 4.1 | 1.8 | 0.7 |

### Example 13

### Effect of Formulation Composition on the Process Recovery of Adjuvanted Protein Antigen Vaccine

Recombinant sub-unit protein antigen of molecular weight 83 kDa was formulated in the presence of aluminum-based adjuvant. 0.2 mg/mL of the protein antigen was initially mixed with 1.5 mg/mL aluminum (Alum) for 30 min and then the formulation components were added, resulting in the formulation composition shown in Table 15. The resulting mixture was spray dried using a Buchi 190 mini-spray dryer equipped with an ultrasonic nozzle, at low pressure, and under the condition of: 0.25 mL/min solution feed rate, 65° C. inlet temperature, and 50° C. outlet temperature. The powders were aliquoted into vials inside of a chamber with controlled humidity and temperature, at <10% RH and 25° C., respectively. The dried samples were reconstituted with double-filtered deionized water and the remaining activities were determined using a cell-based assay to assess the inhibitory capability of the spray dried, adjuvanted vaccine against a recombinant toxin (Table 15).

TABLE 15
Formulation composition and process-associated loss for spray dried Alum-adjuvanted protein
antigen vaccine

| Components¹ | P1 | P2 | P3 | P4 | P5 | P6 | P7 | P8 | P9 | P10 | P11 | P12 |
|---|---|---|---|---|---|---|---|---|---|---|---|---|
| Trehalose | | | | | | 4.0 | 4.0 | 3.9 | 3.9 | | | |
| Sucrose | 4.0 | 4.0 | 4.0 | 3.9 | 3.9 | | | | | 4.0 | 3.8 | 4.0 |
| Raffinose | | | | | | | | | | | | |
| Glycerol | | | | 0.1 | | | | | | | 0.2 | |
| Sorbitol | | | | | 0.1 | | | 0.1 | 0.1 | | | |
| Polysorbate 80 | | | | | | | | | | | | |
| Arginine | | | | | | | | | | | | |
| NaCl | | | 0.07 | | | | | | | | | |
| CaCl₂ (mM) | | | | | | | | | | 2 | 2 | 2 |
| NaPO₄ (mM) | | 5 | | | | | 5 | | 5 | 10 | 10 | |
| Tris (mM) | 20 | | 20 | 20 | 20 | 20 | | 20 | | | | |
| pH | 7.4 | 7.4 | 7.4 | 7.0 | 7.4 | 7.4 | 7.4 | 7.4 | 7.4 | 7.4 | 7.4 | 7.4 |
| Process Loss (%)² | 32.1 | 43.9 | 0.0 | 6.0 | 15.3 | 0.0 | 4.7 | 11.7 | 25.7 | 0.0 | 0.0 | 0.0 |

¹Component composition given as % (w/v), unless stated otherwise
²Calculated process loss based on pre- and post-cavitation dried activity, as determined by the cell-based assay examining the activity of dmPA in inhibiting a lethal factor

What is claimed is:

1. A dry vaccine composition prepared with a formulation, the composition comprising:
a) a biologically active sample;
b) a polyol that, in the formulation, is about 5% to about 70% (w/v);
c) a divalent cation that, in the formulation, is about 0.1 mM to about 100 mM;
d) a plasticizer of 1% to 10% (w/w) in the formulation; and
e) an amino acid, a polymer, or surfactant, or any combination thereof;
wherein the dry vaccine composition is prepared by spray drying.

2. The composition of claim 1, wherein the biologically active sample is a virus at a titer ranging from about 3 log per mL to about 9 log per mL, wherein the titer is determined on the liquid-reconstituted vaccine or determined in the liquid form before processing to make the dry vaccine composition.

3. The composition of claim 1, wherein the biologically active sample is bacteria or an immunologically active oligopeptide or immunologically active oligonucleotide.

4. The composition of claim 1, wherein the biologically active sample is a bacterium.

5. The composition of claim 1, where in the biologically active sample is a bacterium that is Salmonella typhi.

6. The composition of claim 1, wherein the biologically active sample is an immunogenic oligopeptide or oligonucleotide.

7. The composition of claim 1, wherein the biologically active sample is a sub-unit protein antigen, or that is an oligopeptide or oligonucleotide that can induce an immune response against protective antigen (PA).

8. The composition of claim 1, wherein the biological active sample is a virus with a measurable titer, wherein the titer is determined by tissue culture infective dose (TCID) assay or by fluorescent focus assay (FFA).

9. The composition of claim 1 wherein the virus is measles virus, and the formulation contains less than 50 mM pharmaceutically acceptable buffer.

10. The composition of claim 1 wherein the virus is measles virus, and the formulation contains less than 50 mM potassium.

11. The composition of claim 1 wherein the virus is measles virus, and the formulation contains less than 10 mM pharmaceutically acceptable buffer.

12. The composition of claim 1 wherein the virus is measles virus, and the formulation contains less than 10 mM potassium.

13. The composition of claim 1, wherein the virus is adenovirus, and the formulation contains greater than 100 mM potassium.

14. The composition of claim 1 wherein the formulation contains between 0.1-10 mM calcium and 0.1-10 mM zinc.

15. The composition of claim 1, that is prepared with a first formulation that contains 0.1 mM to about 50 mM calcium and 0.1 mM to about 50 mM zinc, and wherein there is a second formulation that contains the same components, at the same concentrations, as the first formulation, but where the second formulation has no zinc, and where the stability of the dried vaccine composition prepared from the first formulation is at least 3.0-fold greater than that of a dried vaccine composition with the same virus, but prepared with the second formulation.

16. The composition of claim 15, wherein the fold greater is, at least 5.0-fold greater.

17. The composition of claim 15, wherein the stability is process stability.

18. The composition of claim 15, wherein the stability is storage stability.

19. The composition of claim 1, that is prepared with a first formulation that contains 0.1 mM to about 50 mM calcium, and 0.1 mM to about 50 mM zinc, and wherein there is a second formulation that contains the same components, at the same concentrations, as the first formulation, but where the second formulation has no calcium, and where the stability of the dried vaccine composition prepared from the first formulation is at least 3.0-fold greater than that of a dried vaccine composition with the same virus, but prepared with the second formulation.

20. The composition of claim 19, wherein the fold-greater is at least 5.0-fold greater.

21. The composition of claim 19, wherein the stability is process stability.

22. The composition of claim 19, wherein the stability is storage stability.

23. The composition of claim 1, where the formulation has greater than 10% solids and less than 40% solids.

24. The composition of claim 1, where the formulation has greater than 15% solids and less than 40% solids.

25. The composition of claim 1, where the formulation has greater than 20% solids and less than 40% solids.

26. The composition of claim 1, wherein the virus is a live attenuated virus.

27. The composition of claim 1 wherein the virus is an enveloped virus.

28. The composition of claim 1, wherein the virus is a non-enveloped virus.

29. The composition of claim 1, wherein the virus is a measles virus or a rotavirus.

30. The composition of claim 1, wherein the polyol is sucrose, trehalose, or a mixture of sucrose and trehalose.

31. The composition of claim 1, wherein the divalent cation is calcium, zinc, magnesium, or a combination thereof

32. The composition of claim 1, wherein the amino acid is alanine, arginine, methionine, serine, lysine, histidine, glutamic acid, or a combination thereof.

33. The composition of claim 1, wherein the formulation comprises a pharmaceutically acceptable buffer.

34. The composition of claim 1, wherein the plasticizer is glycerol, dimethylsulfoxide (DMSO), propylene glycol, ethylene glycol, oligomeric polyethylene glycol, sorbitol, or a combination thereof.

35. The composition of claim 1, wherein the polymer is gelatin, partially hydrolyzed gelatin, albumin, or a combination thereof.

36. The composition of claim 1, wherein the surfactant is polyethylene glycol, polypropylene glycol or a surfactant with the chemical structure of Pluronic® F68.

37. A method for preparing the composition of claim 1 that comprises combining or mixing a formulation with the virus, and then spray drying wherein the pressure (Pₐₜₘ) is less than 25 psi, or wherein the temperature (Tₒᵤₜ) is less than 60 degrees C., or wherein the pressure is less than 25 psi and the temperature is less than 60 degrees C.

38. A method for preparing the composition of claim 1 that comprises combining or mixing a formulation with a virus, and then spray drying wherein the pressure (Pₐₜₘ) is about 10-20 psi, or wherein the temperature (Tₒᵤₜ) is about 30-50 degrees C., or wherein the pressure is about 10-20 psi and the temperature is about 30-50 degrees C.

39. The dry vaccine composition of claim 1, comprising:
a) live attenuated strain of a measles virus at a titer ranging from about 3 Log TCID₅₀/mL to about 9 Log TCID₅₀/mL;
b) a mixture of sucrose and trehalose, each present at a concentration ranging from about 5% to about 20% (w/v);
c) potassium phosphate at a concentration ranging from about 10 mM to about 100 mM adjusted to pH of about 6 to 7;
d) a mixture of calcium chloride and zinc chloride, each present at a concentration ranging from about 1 mM to about 10 mM;
e) arginine at a concentration ranging from about 1% to about 8% (w/v);
f) glycerol at a concentration ranging from about 0.1% to about 5% by weight of said formulation; and
g) human serum albumin at a concentration ranging from about 1% to about 10% (w/v).

40. A formulation of Table 3, Table 5, Table 6, Table 8, Table 9, Table 11, Table 13, Table 14, or Table 15.

41. A dry vaccine composition made from
a virus and a first liquid formulation,
wherein the first liquid formulation contains calcium and zinc,
wherein the dry vaccine composition is a first dry vaccine composition,
wherein the stability of the first dry vaccine is greater than the stability of a second dry vaccine,
wherein the second dry vaccine is prepared with the same virus, same formulation components, and same formulation concentrations, as used for the first dry vaccine composition, except that the second liquid formulation does not contain any calcium, or does not contain any zinc.

42. The dry vaccine composition of claim 41, wherein the stability that is greater, is at least 3.0-fold greater than that of the second dry vaccine.

43. The dry vaccine composition of claim 41, wherein the stability that is greater, is at least 5.0-fold greater than that of the second dry vaccine.

44. The dry vaccine composition of claim 41, wherein the virus is measles virus.

45. The dry vaccine composition of claim 41, wherein the first liquid formulation contains a pharmaceutically acceptable buffer.

46. The dry vaccine composition of claim 41, wherein the first liquid formulation contains one or more of a polyol, an amino acid, a plasticizer, a polymer, a surfactant, or any combination thereof.

47. The dry vaccine composition of claim 41, wherein the stability is process stability.

48. The dry vaccine composition of claim 41, wherein the stability is storage stability.
